(19) United States
(12) Patent Application Publication
(10) Pub. No.: US 2008/0045475 A1
(43) Pub. Date: Feb. 21, 2008

(54) Elemental cellular therapy is a genetic, cellular and disease-modifying therapy which enhances the systemic conduct of genetic and cellular transmethylation activity resulting in enhancement of concerted genetic and cellular metabolic, physiologic and homeostatic processes

(76) Inventor: Phillip Edward Littmann, Cincinnati, OH (US)

Correspondence Address: PHILLIP E. LITTMANN, 11028 ALLENHURST BLVD EAST, CINCINNATI, OH, 45241, US

(21) Appl. No.: 11/506,500

(22) Filed: Aug. 20, 2006

(51) Int. Cl. A61K 31/714; A61K 31/7008; A61K 31/525; A61K 38/46; A61K 36/906; A61K 31/095

(52) U.S. Cl. 514/52; 514/62; 514/250; 514/251; 514/706; 514/27; 514/456; 424/94.65; 424/756

(57) ABSTRACT

Methal sulfonyl methane, glucosamine sulfate, folic acid and vitamin B-12 composition for the enhancement of the execution of transmethylation activity within every living cell in the body for humans and all other members of every other genus and species in the entire Animal Kingdom.

Elemental Cellular Therapy is a genetic, cellular and disease-modifying therapy which enhances the systemic conduct of genetic and cellular transmethylation activity resulting in enhancement of concerted genetic and cellular metabolic, physiologic and homeostatic processes.

### 1. FIELD OF THE INVENTION

The present invention is a novel integrated system of therapy designated as Elemental Cellular Therapy or ECT. It has been enabled as devised entirely on the basis of discoveries made in a progressive manner in the course of the evolution of the development of this therapy involving new fundamental knowledge as well as unprecedented insights with respect to principles and processes operative in the essential nature of molecular and sub-molecular activity involving human and animal genetics, immunology, biochemistry, cellular biology and the pathophysiology of disease. It involves the development of what has been defined as the “core regimen” the pharmacological activity of which alters the fundamental molecular and sub-molecular context in which life itself proceeds at a genetic and cellular level of activity. This in and of itself enhances the fundamental nature and therapeutic activity of all existing pharmacological therapy in such a way as to make each a new and entirely novel therapeutic intervention exclusive of its novel interactivity in a modified genetic and cellular milieu. The evolutionary development and implementation of ECT has ultimately resulted in the establishment of an entirely novel and radically different paradigm for the concept of what constitutes a disease and the optimal manner in which it may be treated. The concept and development of the core regimen and subsequently that of ECT includes an appreciation of the most proximate cause and the least common denominator responsible for the fundamental expression of disease activity. This involves the unique molecular and sub-molecular biological activity that is responsible for the expression of what have been discovered in nature to exist as discretely unique essential pathological activities or influences. Each of these is defined in this document in terms of the mutually exclusive nature of the molecular and sub-molecular biological activity that is unique of each from one another. Disease activity is described herein in an entirely radical conceptual manner as the concerted systemic and interdependent interactivity of two or more of what have been determined and defined to be to be essential pathophysiological influences. Each of these is known to exists in a mutually exclusive manner as constituting one of a discrete number of such influences the synergistic and interdependent interactivity of two or more of which is capable of being descriptive and expressive of the pathophysiology of all disease afflicting humans. The therapeutic basis of ECT involves an appreciation of all of the aforementioned. The development of novel genetic and cellular disease modifying therapy constituting ECT has involved the strategic application of the laws of physics and the principles of analytic chemistry and its stochoiometry to devise novel genetic, cellular and disease modifying therapies the pharmacological activity of which may be expressed in terms of the unique molecular and sub-molecular biological activity of each.

Given an appreciation of the essential molecular and sub-molecular biochemical activity responsible for the expression of each essential pathophysiological disease influence, genetic, cellular and disease modifying therapy has been strategized with an appreciation for the predictable influence of the of the molecular and sub-molecular biological activity definitive of each of the disease-focused permutations of ECT and the unique molecular and sub-molecular biological activity that is definitive of each essential pathophysiological influence. The pharmacological influence of the concerted molecular and sub-molecular biological activity of this therapy exerts its unique genetic, cellular and disease modifying influence upon the conduct of the molecular and sub-molecular biological activity of each of the essential pathophysiological influences involved in a given disease thereby altering the fundamental activity of the disease process itself; inclusive of the “aging processes”. The result is that if all of the essential pathophysiological influences defined in this patent can be effectively addressed in a molecular and sub-molecular disease modifying manner, the efficacy of ECT in the genetic and cellular disease modifying therapy of any disease process may be successfully achieved 100%.

### 2. BACKGROUND OF INVENTION

Elemental Cellular Therapy (or ECT) is an integrated system of genetic, cellular and disease modifying therapy. The pharmacological activity through which the majority of therapeutic benefits that proceed from the administration of every dose of the core regimen of ECT is achieved through the systemic enhancement of concerted genetic and cellular transmethylation. The pharmaceutical agents comprising the core regimen comprise the foundation upon which all of the disease-focused pharmaceutical permutations of therapy with ECT have been developed. It also involves the use of prescription and non-prescription pharmaceutical agents or compounds configured in prescribed regimens that involve utilization of the pharmacological activity of each agent or compound in novel synergistic and mutually interdependent interactivity with one another.

ECT provides therapeutic benefits in a concerted and essentially equivalent fashion for every living cell in the body of a treated individual of the designated target population for whom this therapy has been devised. The “target population” includes al humans and all members of every other genus and species in the entire Animal Kingdom. There are no exceptions to the cellular distribution of benefits within the organ systems of the body, including the brain and peripheral nervous system. The therapeutic activity provided with the use of this therapy literally alters the manner with which each and every cell in the body responds to disease, injury, various stresses and even “aging”.

Core Regimen

ECT involves the use of a “core regimen” along with complementary therapies. The pharmacological activity of the core regimen alone provides unprecedented modification, modulation and augmentation of the genetic and cellular metabolic, physiologic and homeostatic activity of each and every cell in the body. Benefits provided from the clinical use of the core regimen alone include but are not limited to:
Enhanced genetic and cellular replication and repair the conduct and degree of which significantly exceeds the inherent limits placed upon the optimal execution of these activities in nature
Enhanced protein synthesis (i.e. genetic expression) the conduct and degree of which also substantially exceeds the inherent limits place upon the optimal execution of these activities in nature
Re-establishment of genetic expression/protein synthesis that has previously ceased altogether as a consequence of a facet of a disease process or the “aging” process
Enhanced activity of global intrinsic immune response to a degree and in a manner that exceeds the inherent limits placed upon human and animal immune response by nature itself/An example of how this benefit proceeds with respect to the treatment, prevention and suppression of all viral disease involves the enhancement of endogenous interferon and its requisite activation as well as the replication of lymphocytes and their subsequent activation. Among the viral diseases with which this therapy has been proven effective include rhinoviruses (colds), influenza, herpes simplex, herpes zoster, hepatitis A, hepatitis B, including chronic hepatitis B/ all of the aforementioned processes proceed from the enhancement of genetic and cellular transmethylation. Enhanced transmethylation activity is also responsible in corresponding fashion for remarkable enhancement of treatment and prevention of bacterial infections
Down-regulation of intrinsic auto-immune activity/this proceeds more or less concurrently with the augmentation of intrinsic immune response.

Transmethylation Activity

The singular and unprecedented pharmacological activity that is exerted following every dose of the core regimen includes but is not entirely limited to the systemic enhancement of concerted genetic and cellular transmethylation activity. Described in more detail and context below, transmethylation reactions constitute the requisite rate limiting step executed in the completion of the qualitative and quantitative completion of all essential genetic and cellular biochemical reactions. The method for this incomparable pharmacological activity is accomplished through the systemic enhancement of the concerted genetic and cellular transmethylation activity. In nature the qualitative and quantitative completion of essential genetic and cellular biochemical processes are known to be accomplished in a teleologically constant, invariably fixed and perpetual fashion within each and every living cell countless times daily throughout the entire body for all humans. This is a changeless immutable phenomenon the nature of which in essence is reflective of the fundamental quality of the life for every human being on the planet. Fundamental concepts involving genetic and cellular molecular and sub-molecular biologic activity are definitive of the teleologic relationship between the completion of essential genetic and cellular biochemical reactions and the performance of genetic and cellular processes. The qualitative and quantitative activity of the completion of all genetic and cellular biochemical reactions proceeds in a directly proportional and congruent manner with the nature of the performance of genetic and cellular metabolic, physiologic and homeostatic processes. It is therefore implicit that the fundamental nature of the performance of essential genetic and cellular processes proceeds in the same teleologically constant, invariably fixed and perpetual fashion within each and every living cell daily throughout the entire body for all humans.

The influence in nature that each individual living cell is ultimately able to exert upon the dynamic nature of the performance of essential genetic and cellular metabolic, physiologic and homeostatic processes is none at all. These reactions proceed to completion in compliance with the same laws of physics and analytic chemistry and its stochiometry as does the reaction that is observed to proceed when baking soda is added to a beaker of vinegar. Since it is implicit that completion of all essential genetic and cellular biochemical processes proceed countless times a day and in a perpetual manner within each and every living cell in the body, there must be a teleological influence of some kind preventing the completion of all genetic and cellular biochemical reactions, and therefore the performance of all of the mated biochemical processes, from grinding to a halt. This would otherwise reasonably be expected to occur readily as the rate limiting participant involved in the requisite execution of the rate limiting step completed among all others in the completion of each and every biochemical reaction is entirely consumed as it is predictably in the completion of analogous chemical reactions.

However there is an essential and fundamental difference between chemical and biochemical reactions. The rate-limiting participant in the requisite execution of the rate-limiting step involved in completing the chemical reaction involving baking soda and vinegar is the baking soda. Regarding the fundamental nature of genetic and cellular molecular and sub-molecular biochemical activity, the rate limiting participant in the execution of the requisite rate-limiting step that is accomplished among all others involved in the completion of each and every essential genetic and cellular biochemical reaction proceeding in every cell in the body is actually a sub-molecular particle, a methyl moiety (CH3). The execution of this aforementioned rate-limiting step is designated to be transmethylation. Each of these aforementioned genetic and cellular biochemical reactions and therefore all transmethylation activity are known to proceed countless times daily and in a perpetual manner within each and every living cell in the body for humans and all other members of every other genus and species in the entire Animal Kingdom.
The molar concentration of baking soda in the above-mentioned chemical reaction is fixed and of necessity begins to dwindle immediately at the commencement of the reaction predictably to zero as the reaction proceeds.
The teleologically requisite source from which each and every cell in the body acquires methyl moieties for the execution of the rate-limiting step that is accomplished among all others involved in the completion of each and every essential genetic and cellular biochemical reaction is an endogenous anatomically comprehensive but non-descript “fund” of these sub-molecular particles.
The molar concentration of methyl moieties maintained in this fund is maintained in nature throughout the entire body in such a manner that it remains constant, invariantly fixed and immutable throughout life for humans and all other animals of every other genus and species in the Animal Kingdom.
It follows that the execution of all essential genetic and cellular rate-limiting steps is also accomplished of teleological necessity in a constant, invariantly fixed and immutable fashion.
Therefore the same can be said of the performance of essential genetic and cellular biochemical metabolic, physiologic and homeostatic processes with which each of the aforementioned essential biochemical reactions is mated in nature.
It is therefore implicit that the qualitative and quantitative activity of the completion of concerted genetic and cellular biochemical reactions exerts a congruent and directly proportional influence upon the fundamental nature of the performance of essential genetic and cellular metabolic physiological and homeostatic processes with which each is teleologically associated.
Connecting all of the aforementioned “dots”, it follows that in nature the fundamental nature of the systemic performance of concerted essential genetic and cellular biochemical processes are accomplished in a perpetual manner without interruption within each and every cell throughout the entire body. Further this is known of teleoligical necessity to proceed in a constant, invariably fixed and immutable manner throughout the entire life of every human owing in most proximate fashion to the invariability of the molar concentration of methyl moieties that ultimately influences in a directly proportional manner the fundamental performance of essential genetic and cellular metabolic, physiologic processes.
It is therefore implicit that the achievement of the ability to augment the ambient concentration of methyl moieties with in the anatomically non-descript fund of these submolecular particles in the body with conventional, albeit revolutionary, pharmacological therapy is the functional equivalent of achieving discretionary control to enhance the essential quality of life in every cell in the body and therefore for an individual human being, all human beings.

Elemental Pathophysiological Processes Involved in Disease

In the course of the development of ECT I have observed that all human and animal disease activity including the process of human aging itself may be described as the concerted synergistic and interdependent interactivity of two or more of the following elemental pathological influences:
Infection
Inflammation
Auto-immune activity
Pathological cellular hypertrophy
Extinguished genetic expression (i.e. protein synthesis) as a consequence of “aging” or a component of the pathophysiology of disease activity
Pathological activity proceeding from the influence of an imbalance in the central nervous system neurotransmitter axis that includes but is not limited to the activity of serotonin, dopamine and norepinephrine

The enhancement of genetic and cellular transmethylation activity that proceeds in an exclusive fashion with the administration of every dose of the core regimen has been observed reliably to enhance the conduct of intrinsic human and animal immune response and activity. What has also been observed is that a deficiency in the optimal balance of the central nervous system neurotransmitter axis, defined as such in this patent, impairs an optimal response to enhancement of intrinsic immune activity.

In the course of the development of ECT, it has been observed that there exists molecules the biological activity of which are responsible for fundamentally unique inflammatory activity that is an essential component of disease activity in humans and animals. Among these, but not inclusive of these, are histamine, cytokines, leukotrienes, lymphokines and bradykinin. In the course of developing various disease focused pharmaceutical permutations of ECT, it has been observed that some disease processes amenable to genetic, cellular and disease-modifying therapy with ECT do not possess an inflammatory component in their fundamental pathophysiology. These include, but are certainly not limited to, chronic progressive diabetic renal insufficiency, congestive heart failure, left ventricular hypertrophy, diastolic dysfunction, pulmonary hypertension. The majority of disease processes afflicting humans have been observed to have a significant inflammatory component necessitating effective down-regulation in order to provide effective disease-modifying therapy. The down-regulation of the inflammatory component of such diverse disease processes as multiple sclerosis, idiopathic pulmonary fibrosis, dermatomyositis, interstitial cystitis, etc. has been successfully achieved with the use of a regimen of unique anti-inflammatory agents, the pharmacologic activity of each of which acts in synergistic interdependent interactivity with the pharmacologic activity of the core regimen. These include but are not limited to quercetin, curcumin, tumeric, bromelain and ginger. An essential component of effective therapy in down-regulating inflammation as it constitutes an essential pathophyisologic influence is the enhancement of genetic and cellular transmethylation activity. What has also been observed is that a deficiency in the optimal balance of the central nervous system neurotransmitter axis, defined as such in this patent, impairs an optimal response in the down-regulation of inflammation with ECT as a essential pathophysiologic influence.

Auto-immune activity as an essential pathophysiologic influence in human disease has been discovered to be a component in the fundamental pathophysiologic activity of disease processes including multiple sclerosis, idiopathic pulmonary fibrosis and polycystic kidney disease. Unexpectedly, auto-immunity as a pathophysiological influence has also been discovered to be operative in congestive heart failure, left ventricular hypertrophy, diastolic dysfunction, pulmonary hypertension. In the absence of their effective down-regulation, these disease processes do not respond to therapy with ECT. The enhancement of genetic and cellular transmethylation activity that proceeds in an exclusive fashion with the administration of every dose of the core regimen has been observed to down-regulate intrinsic human auto-immune activity in an essentially but not entirely corresponding fashion in which it enhances human and animal immune response. What has also been observed is that a deficiency in the optimal balance of the central nervous system neurotransmitter axis, defined as such in this patent, impairs an optimal response in the down-regulation of intrinsic auto-immune activity with ECT as a essential pathophysiologic influence. The impaired or otherwise dysfunctional molecular and sub-molecular biologic activity involving protein synthesis and the discontinuation or impairment of genetic expression (protein synthesis) as a facet of the pathophysiology of disease or a component of the pathophysiologic activity of “aging” has been determined to constitute an essential pathophysiologic influence in the expression of human disease activity. The enhancement of genetic and cellular transmethylation activity proceeding from the administration of the core regimen alone has been reliably observed to provide therapeutic benefits already described in the aforementioned section entitled Core Regimen. What has also been observed is that a deficiency in the optimal balance of the central nervous system neurotransmitter axis, defined as such in this patent, impairs an optimal response in the pharmacological activity of the core regimen and therefore therapeutic benefits accruing from ECT upon dysfunction in genetic expression and protein synthesis.

For the purposes of this patent, imbalance in the central nervous system neurotransmitter axis is descriptive of an essential pathophysiologic process that is itself influential in impairing the effectiveness of ECT to have an optimal genetic and cellular disease modifying impact upon each one of the other essential pathophysiologic influences described above. The fundamental nature of what constitutes the optimal balance of the aforementioned central nervous system neurotransmitter axis includes but may not be limited to the optimal metabolic activity of serotonin, dopamine and norepinephrine in the central nervous system as well as optimal activity of central nervous system neurotransmission responsible for mood stability. The ability to objectively assess the success of achieving optimal balance of central nervous system neurotransmitter activity or approximating the success is achieved in part by the comparison of serum compliment levels C3 and C4 before and after the initiation of therapy that is influential in improving the quality of a balance in the central nervous system neurotransmitter activity. It has been observed in the course of the development of ECT that serum compliment fraction C3 and C4 are consumed as a result of increased intrinsic auto-immune activity resulting in the reduction in the measure of serum C3 and C4 There exist pharmacologic agents that contribute to the quality of the balance in central nervous system neurotransmitter activity but the use of each alone, including the entirely effective use of each alone, is sometimes not capable of restoring balance in the central nervous system neurotransmitter axis. These include, but are not limited to, Paxil, Zoloft, Wellbutrin, Lexapro, Cymbalta, Prozac and also such drugs as lithium, Lamictal, Depakote, etc. The reason for this is that the individual's response in the case of the therapeutic use of specific selective serotonin reuptake inhibitors, the effective use of which contributes to the improvement of the balance of central nervous system neurotransmitter activity, is entirely unique to that individual. In other words, one individual may have a remarkable and ideal response to therapy with Zoloft while another individual treated with Zoloft may have a marginal response and do much better on another selective serotonin reuptake inhibitor. It is likely that this variant response among individuals is related to differences in the molecular structure of protein carriers. With respect to achieving an optimal balance in the central nervous system neurotransmitter axis that enables an optimal an even sometimes a sub-optimal response to therapy with ECT, it is sometimes necessary for an individual to be treated with a selective serotonin reuptake inhibitor, a dopamine reuptake inhibitor as well as a norepinephrine reuptake inhibitor and/or pharmacologic therapy effective in stabilizing mood imbalance as in bi-polar disease.

The treatment of disease processes and the genetic and cellular structural and functional dysfunction associated with the “aging” process are all capable of being treated in a genetic, cellular and disease modifying and reparative manner. This involves the administration of the core regimen which will act with synergistic and interdependent interactivity with one or more complementary agents or combination of agents. The pharmacological activity of each agent alone or the concerted pharmacological activity of a collection of agents have been strategized to down-regulate, eliminate or otherwise neutralize one or more of these above-mentioned elemental pathophysiological influences.

The concept of ECT providing disease modifying and genetic and cellular reparative therapy for injury and disease as well as the aging process necessarily involves the obligation of the down-regulation, elimination or otherwise neutralization of the above-mentioned fundamental pathological influences.

### 3. DESCRIPTION OF BACKGROUND ART

| | |
|---|---|
| United States Patent | 6,583,123 |
| Henderson, et al. | Jun. 24, 2003 |

Aminosugar, glycosaminoglycan, and S-adenosylmethionine composition for the treatment and repair of connective tissue

| | |
|---|---|
| United States Patent | 5,508,271 |
| Rabinoff | Apr. 16, 1996 |

Treatment of neurological dysfunction with methylcobalamin

| | |
|---|---|
| United States Patent | 6,255,295 |
| Henderson, et al. | Jul. 3, 2001 |

Aminosugar, glycosaminoglycan or glycosaminoglycan-like compounds, and s-adenosylmethionine composition for the protection, treatment, repair, and reduction of inflammation of connective tissue

| | |
|---|---|
| United States Patent | 5,466,678 |
| Kawabata, et al. | Nov. 14, 1995 |

Co-administration of S-adenosyl-L-methionine to reduce the nephrotoxicity of cisplatin therapy

| | |
|---|---|
| United States Patent | 5,059,595 |
| Le Grazie | Oct. 22, 1991 |

Pharmaceutical compositions containing 5-methyltetrahydrofolic acid, 5-formyltetrahydrofolic acid and their pharmaceutically acceptable salts in controlled-release form active in the therapy of organic mental disturbances

| | |
|---|---|
| United States Patent | 5,053,429 |
| Hirsch, et al. | Oct. 1, 1991 |

### 4. REFERENCES

Lehninger Principles of Biochemistry . . . Copyright 1997-2006

Textbook of Medical Physiology: Arthur C Guyton: ISBN 0721602401

### EXAMPLE 1

The composition of the present invention for disease modifying therapy of dermatomyositis of a 70 kg human is administered in four divided doses daily about but not essentially six hours apart. The preferred embodiment of each dose of which includes 1000 mg to 3000 mg of methyl sulfonyl methane, 800 mcg of folic acid, 1000 mcg of vitamin B12 and 1000 mg of glucosamine sulfate. It also includes a dose of an angiotensin converting enzyme inhibitor, for example ramapril, 10 mg administered once daily or dose adequate to achieve an undetectable level of serum angiotensin converting enzyme. It may also include the administration of a serotonin and/or dopamine and/or norepinephrine reuptake inhibitor, in such a manner to successfully promote the optimal balance of the central nervous system neurotransmitter axis. It may also be necessary to employ agents involved in stabilizing imbalance of mood that may include lithium, Lamictal or Depakote. The efficacy of the administration of the latter may be assessed with substantial accuracy through the comparisons of sequential measures of serum complement fractions C3 and C4. It also includes the administration of an anti-inflammatory regimen n four divided doses each of which includes but may not be necessarily limited to quercetin 500 mg, tumeric (standardized to contain 97% curcuminoids) 300 mg., bromelain (2400 gdu/gr) 400 mg., ginger (standardized to contain 5% gingerols) 200 mg.

### EXAMPLE 2

The composition of the present invention for disease modifying therapy of chronic progressive diabetic renal insufficiency in a 70 kg human is administered in four divided doses daily about but not essentially six hours apart. The preferred embodiment of each dose of which includes 1000 mg to 3000 mg of methyl sulfonyl methane, 800 mcg of folic acid, 1000 mcg of vitamin B12 and 1000 mg of glucosamine sulfate. It also includes a dose of an angiotensin converting enzyme inhibitor, for example ramapril, 10 mg administered once daily or dose adequate to achieve an undetectable level of serum angiotensin converting enzyme. It may also include the administration of a serotonin and/or dopamine and/or norepinephrine reuptake inhibitor, in such a manner to successfully promote the optimal balance of the central nervous system neurotransmitter axis. It may also be necessary to employ agents involved in stabilizing imbalance of mood that may include lithium, Lamictal or Depakote. The efficacy of the administration of the latter may be assessed with substantial accuracy through the comparisons of sequential measures of serum complement fractions C3 and C4.

### EXAMPLE 3

The composition of the present invention for disease modifying therapy of congestive heart failure in a 70 kg human is administered in four divided doses daily about but not essentially six hours apart. The preferred embodiment of each dose of which includes 1000 mg to 3000 mg of methyl sulfonyl methane, 800 mcg of folic acid, 1000 mcg of vitamin B12 and 1000 mg of glucosamine sulfate. It also includes a dose of an angiotensin converting enzyme inhibitor, for example ramapril, 10 mg administered once daily or dose adequate to achieve an undetectable level of serum angiotensin converting enzyme. It may also include the administration of a serotonin and/or dopamine and/or norepinephrine reuptake inhibitor, in such a manner to successfully promote the optimal balance of the central nervous system neurotransmitter axis. It may also be necessary to employ agents involved in stabilizing imbalance of mood that may include lithium, Lamictal or Depakote. The efficacy of the administration of the latter may be assessed with substantial accuracy through the comparisons of sequential measures of serum complement fractions C3 and C4.

### EXAMPLE 4

The composition of the present invention for disease modifying therapy of HIV/AIDS in a 70 kg human is administered in four divided doses daily about but not essentially six hours apart. The preferred embodiment of each dose of which includes 1000 mg to 3000 mg of methyl sulfonyl methane, 800 mcg of folic acid, 1000 mcg of vitamin B12 and 1000 mg of glucosamine sulfate. It also includes a dose of an angiotensin converting enzyme inhibitor, for example ramapril, 10 mg administered once daily or dose adequate to achieve an undetectable level of serum angiotensin converting enzyme. It may also include the administration of a serotonin and/or dopamine and/or norepinephrine reuptake inhibitor, in such a manner to successfully promote the optimal balance of the central nervous system neurotransmitter axis. It may also be necessary to employ agents involved in stabilizing imbalance of mood that may include lithium, Lamictal or Depakote. The efficacy of the administration of the latter may be assessed with substantial accuracy through the comparisons of sequential measures of serum complement fractions C3 and C4.

What is claimed is:

1. A composition for the systemic enhancement of the concerted conduct of genetic and cellular transmethylation activity in humans and animals constituting the pharmacological activity of the core regimen and comprising the administration in a prescribed manner of a combination of pharmaceutical agents including methyl sulfonyl methane, glucosamine sulfate, folic acid and vitamin B-12, as well as the administration of the composition of claim 2.

2. A therapeutic method contributing to the successful achievement of an optimal balance in the central nervous system neurotransmitter axis of humans, the imbalance of which constitutes an essential pathophysiologic influence responsible for disease activity in humans and animals. This involves the administration of one or more pharmaceutical agents each of which provide a therapeutic influence upon the dysfunction of one or more of the following: serotonin neurotransmitter activity, dopamine neurotransmitter activity and norepinephrine neurotransmitter activity in the central nervous system It also may include the administration of one or more of a diversity of pharmaceutical agents the use of which is known to be effective in the disease modifying therapy of individuals afflicted with a pathologic condition of instability of mood, including but not limited to individuals with bi-polar disease and to a lessor degree with ADHD, as well as the administration of the composition of claim 1.

3. A therapeutic method for the effective down-regulation of inflammation that constitutes another essential pathophysiologic influence responsible for disease activity. This involves the administration of anti-inflammatory agents having a specific pharmacologic activity upon the aforementioned inflammation and includes, but may not be limited to, a prescribed dose of quercetin, turmeric, bromelain and ginger. This also involves the administration of the composition of claim 1 as well as the administration of the composition of claim 2.

4. A therapeutic method for the effective down-regulation of intrinsic auto-immune activity constituting another essential pathophysiologic influence responsible for disease activity. This proceeds with the administration of the composition of claim 1 alone or the administration of the composition of claim 2 alone and in an optimal fashion with the administration of the composition of claim 1 as well as the composition of claim 2.

5. A therapeutic method for the effective down-regulation of pathologic cellular hypertrophy constituting another essential pathophysiologic influence responsible for disease activity. This involves the administration of angiotensin converting enzyme inhibitor agents for the purpose of reducing the measure of serum angiotensin converting enzyme to undetectable levels. This also involves the administration of the composition of claim 1 as well as the administration of the composition of claim 2.

6. A therapeutic method for the effective re-establishing of genetic expression, i.e. protein synthesis proceeding as a facet of pathophysiologic activity responsible for disease as well as “the aging process” and constituting another essential pathophysiologic influence responsible for disease activity. This also involves the administration of the composition of claim 1 as well as the administration of the composition of claim 2.

7. A therapeutic method for the effective enhancement of intrinsic human and animal immune response and activity with respect to every kind of viral and bacterial infectious agent and likely to some degree with respect to infection including prions (thought to be infectious proteins) and parasitic infestation.

8. A method for monitoring in an objective fashion the success with which claim 1 through claim 7 are accomplished in a principal manner involving the measure of serum complement fractions C3 and C4

9. A method for evaluating the success with which the activity of serum angiotensin converting enzyme is reduced to undetectable levels of necessity in a requisite manner for optimal reduction and reversal of pathologic cellular hypertrophy in its role as an essential pathophysiologic influence.

10. A method for the down-regulation of the activity responsible for elevated measures of C-reactive protein (CRP) to undetectable levels and the maintenance thereof.

11. The composition of claim 1 wherein a dose of methyl sulfonyl methane ranges from about 4,000 mg to 12,000 mg or more depending upon the clinical application involved and administered in divided doses four times daily for a 70 kg adult. Proportionate dosing for individuals of lesser or greater weights may necessitate some adjustment. This agent may be administered in any one or more of a number of routes including, but not limited to, oral, G-tube, continuous or intermittent intravenous infusion, enteral and aerosolized for inhalation. It also maybe diluted appropriately for nasal and intraoccular therapy and compounded in appropriate concentrations with existing vehicles for topical administration alone or in combination with other agents.

12. The composition of claim 1 wherein a dose of glucosamine sulfate ranges from about 4,000 mg to 8,000 mg or more depending upon the clinical application involved and administered in divided doses four times daily for a 70 kg adult. Proportionate dosing for individuals of lesser or greater weights may necessitate some adjustment. This agent requires biophysiologic processing in the gut for the production of plasma proteins, therefore administration is limited to oral and G-tube routes.

13. The composition of claim 1 wherein a dose of folic acid ranges from about 3,200 mcg to 6,400 mcg or more depending upon the clinical application involved and administered in divided doses four times daily for a 70 kg adult. Proportionate dosing for individuals of lesser or greater weights may necessitate some adjustment. This agent may be administered in any one or more of a number of routes including, but not limited to, oral, G-tube, continuous or intermittent intravenous infusion, enteral and aerosolized for inhalation. It also maybe diluted appropriately for nasal and intraoccular therapy and compounded in appropriate concentrations with existing vehicles for topical administration alone or in combination with other agents.

14. The composition of claim 1 wherein a dose of vitamin B-12 ranges from about 4,000 mcg to 8,000 mcg or more depending upon the clinical application involved and administered in divided doses four times daily for a 70 kg adult Proportionate dosing for individuals of lesser or greater weights may necessitate some adjustment. This agent may be administered in any one or more of a number of routes including, but not limited to, oral, G-tube, continuous or intermittent intravenous infusion, enteral and aerosolized for inhalation. It also maybe diluted appropriately for nasal and intraoccular therapy and compounded in appropriate concentrations with existing vehicles for topical administration alone or in combination with other agents.

15. The composition of claim 2 wherein the pharmaceutical agents of classes having an influence upon the promotion of the normalization of serotonin, dopamine and norepinephrine neurotransmitter activity in the central nervous system. These drugs include but are not limited to, Paxil, Zoloft, Wellbutrin, Lexapro, Cymbalta, Prozac and also such drugs promoting the stability of mood as described in claim 2. These latter include but are not limited to lithium, Lamictal, Depakote, etc.

16. The composition of claim 3 wherein the average dose of quercetin for a 70 kg human is about 500 mg four times daily. Proportionate dosing for individuals of lesser or greater weights may necessitate some adjustment.

17. The composition of claim 3 wherein the average dose of turmeric for a 70 kg human is 300 mg four times daily. Proportionate dosing for individuals of lesser or greater weights may necessitate some correction.

18. The composition of claim 3 wherein the average dose of bromelain for a 70 kg human is about 400 mg four times daily. Proportionate dosing for individuals of lesser or greater weights may necessitate some correction.

19. The composition of claim 3 wherein the average dose of ginger extract for a 70 kg human is about 200 mg four times daily. Proportionate dosing for individuals of lesser or greater weights may necessitate some correction.

20. The composition of claim 4 includes the administration of composition of claim 1 as well as the composition of claim 2.

21. The composition of claim 5 wherein the dose of any one or a combination of two or more angiotensin converting enzyme inhibitors are administered in such a manner as to reduce and maintain the measure of serum angiotensin converting enzyme at an undetectable level. Examples of this class of agents includes but are not limited to ramapril, lisinopril, benazepril or quinapril.

22. The composition of claim 6 includes the administration of composition of claim 1 as well as the composition of claim 2.

23. The composition of claim 7 includes the administration of composition of claim 1 as well as the composition of claim 2.

24. The method for claim 8 involves a pre-treatment measure of serum complement levels C3 and C4 for subsequent comparison with levels drawn in the course of therapy with ECT, a positive therapeutic response being a decrease in the consumption of serum complement fractions C3 and C4 as a result of down-regulation of intrinsic auto-immune activity.

25. The method for claim 9 involves an initial baseline measure of serum angiotensin converting enzyme and subsequent measure of the same to assess progress with effective therapy in reducing pathologic cellular hypertrophy as well as promoting an optimal balance of central nervous system neurotransmitter activity.

26. The method for claim 10 involves the administration of the composition of claim 1 as well as the composition of claim 2.
